(19) United States
(12) Patent Application Publication
(10) Pub. No.: US 2022/0362431 A1
(43) Pub. Date: Nov. 17, 2022

(54) Visible and infrared light reflective air purification system

(71) Applicant: Ariel Efrati, Jerusalem (IL)

(72) Inventor: Ariel Efrati, Jerusalem (IL)

(21) Appl. No.: 17/741,195

(22) Filed: May 10, 2022

(30) Foreign Application Priority Data
May 11, 2021 (GB) GB2106680.8

(51) Int. Cl. A61L 9/18; A61L 2/26; A61L 2/08; C02F 1/30

(52) U.S. Cl. CPC A61L 9/18; A61L 2/26; A61L 2/084; A61L 2/085; A61L 2/088; C02F 1/30; A61L 2209/12; A61L 2209/15; A61L 2209/16; A61L 2202/11; C02F 2303/04

(57) ABSTRACT

A versatile air purification system is disclosed herein. The invention harnesses reflective light trapping components capable of circulating high-intensity visible and infrared radiation. Passing air absorbs a part of the energy in aerosols as viruses and bacteria. Excessive energy consequently induces denaturation of biomacromolecules and can trigger chemical and physical disinfection mechanisms to airborne pathogens. This technology is also compatible with various HVAC systems, like those of medical facilities, buildings, and vehicles, and can be utilized for other fluids too. The system can further be applied as a distinct, portable air purification apparatus.

### FIELD OF THE INVENTION

The present invention relates to pathogen purification of air in HVAC (heat ventilation air conditioning) systems, through trapped low frequency radiation.

### BACKGROUND OF THE INVENTION

Air transported pathogens impose a growing concern, particularly in hospitals, hotels and public transportation systems as commercial flights and cruise ships. Recent COVID-19 pandemic spread is suspected to be fueled in part through HVAC systems airborne transmission. While public health damages are evident, and financial losses are vast, further development of means of improving air quality is essential.

In order to achieve this goal, several inventions attempted to disinfect flowing air through ventilation systems. Most prior art harnessed the high energetic ultra-violet (UV) light as means of disinfection. Typically, a photon in the UV range possesses the energy capable to break down covalent chemical bonds. When such photon is absorbed by a molecule in a pathogen, as a protein or genetic material such as DNA or RNA, it may chemically alter or break down the molecule into a dysfunctional form. Some prior art promoted the magnification of the electromagnetic field through reflectance. Englel, S. et al, U.S. Pat. No. 7,976,195 B2 ; Liu, B. D. U.S. Pat. No. 8,772,744 B1 and Clark, R. W. U.S. Pat. No. 8,404,186 B2 all harnessed reflection of UV light as means of disinfection. By that principle, the light is being multiplied without additional power or light sources. That technology was proven effective especially in water disinfection devices as in Cooper, J. R. et al. US 2018/0055956 A1. Moreover, Tribelsky, Z. et al US RE43,332 E used the same principles over liquids in addition to air, to reflect any type of light for disinfection. This patent focuses on the physical funneling of the flowing material to a narrower outlet. It also suggests focusing light onto the condensed material through parabolic reflectors (As in Hed, A. Z U.S. Pat. No. 5,727,108). An opposing approach was taken by Blechschmidt, J. et al. U.S. Pat. No. 9,999,696, B2, increasing the homogeneity of the reflected electromagnetic field by a channel with planar reflectors. According to this invention, UV or infrared radiation is reflected from plains which are positioned in parallel to the average flow direction, for the increase of light homogeneity. Matschke, A. L. developed a uniformly UV reflective duct for cleansing of both air and water. The main embodiment of this invention allows the three-dimensional retention of UV light through an ellipsoidal duct. More exotic utilitarian instruments for sanitation extend from microwave combinations (Thompson, G. US 2004/0262298 A1) and up to light absorbing physical network, for air heating effect (Eduardo, D. B. et al. WO 2011/119525 A2).

### SUMMARY OF THE INVENTION

All pathogens, including viruses, bacteria, fungi, and some forms of algae are sensitive to large doses of electromagnetic radiation. Different types of radiation differ by the sterilization mechanisms and effects over the pathogens. Proteins may undergo denaturation, out from their tertiary structure due to overheating. Even a very short period of overheating has been proved to be effective for sterilization. A single infrared photon is harmless for a pathogen, yet its energy is absorbed and elevates the temperature. If that energy does not completely dissipate before another photon arrives, the temperature will rise even further. Pathogens can absorb infrared radiation directly by some of their molecules. As polyamides, proteins absorb around the wavenumber of 3350 cm⁻¹ (corresponding to a wavelength of 2985 nm) for N-H bond stretching. Around 1680 cm⁻¹ (5952 nm) C═O bond stretching takes place. Additional infrared absorption peaks of proteins, fatty acids, and genetic material are placed in the infrared range. Although visible light absorption among these biomolecules is relatively low, it can induce deactivation among pathogens by different mechanisms. Researches (as Lipovsky A. et al. Lasers in surgery and medicine 42(6) 2010) presented evidence that high-intensity visible light is phototoxic to bacteria. Particularly blue light was found to stimulate the production of cytotoxic reactive oxygen species by bacteria. Additionally, airborne viruses have been shown to be sensitive to ambient humidity (Yang W. and Man L. C. Applied and environmental microbiology 78(19) 2012). Water readily absorb infrared radiation in various wavelengths. Heating or evaporating water that encompass an airborne virus may trigger its inactivation.

It is an object of the current invention to disclose a device and a method aimed to substantially reduce the transmittance of pathogens through HVAC systems, by low wavelength radiation trapping. HVAC refers herein to an air flowing system that possesses any one or more of the following features: heating, ventilating, air conditioning, and cooling. Ventilating according to the current invention is defined to be directing or cycling air from any source e.g. air from the inside, outside or any combination of the two. The chances of a radiated pathogen, such as a virus, to weaken enough to be non-infectious, depend on some radiation factors besides its wavelength. Light intensity and time of exposure play an important role in the deactivation process.

One of the most distinctive features of the current invention is the implementation of high wavelength light trapping devices. In order to achieve a high electromagnetic field capable of pathogen disinfection, reflection can be utilized. Reflection of light by the sides of a duct may redirect a light beam back and forth a few times. Yet, the usage of two or three light sources instead of one, offers a comparable function, without the necessity of a reflective light apparatus. Multiple reflections effect, however, is comparable to the work of the same number of light sources without any reflectance. Placing a big number of light sources inside an HVAC system is impractical. What compounds this solution further is the fact that several light sources insert a lot of energy into the system. This energy input can impose safety issues as well as temperature rise to the outgoing airflow. Nonetheless, intensified flux of photons inside a chamber, due to reflection, should not change the overall energy balance of the system. The reason behind this is the fact that no additional energy input is applied by the reflectors themselves. Since the incoming air to the system is substantially transparent to a wide range of wavelengths, most radiation should eventually be absorbed either by the reflective surfaces or by aerosols as pathogens. Another route for the energy to flow, which should be reduced to the minimum by the current invention is light escape through the inlet or outlet apertures of the light trap. The current invention is, therefore, environmentally friendly and energy-efficient technology, owing to the relatively low energy input that is enhanced by the underlying light enhancing mechanism.

The value of reflectance (r) expresses the probability of a photon to be reflected back from the surface. How many times can light be reflected back and forth inside a trap surrounded mostly by mirrors? The average light path through the trap depends upon several factors, including the light source(s) position, structure and geometry, as well as microscopic scale surface features and air apertures. Let the light be influenced solely by reflectance, the number of paths through the trap can be compared to a converging infinite geometric series:

$${\sum }_{k = 0}^{∞} {r}^{n} = \frac{1}{1 - r}$$

Where r is the reflectance, which is positive and smaller than one. The addition of air to the trap would absorb a part of the light by its aerosols. This process is comparable to a reduction in the chances of photons to be reflected and therefore should increase the denominator in the equation. Similarly, additional escape probability from the inlet and outlet apertures would increase the denominator and decrease the total result. Absorption by aerosols is a favorable reaction for disinfection, which depends on the aerosol content, while light escape from the apertures is unfavorable. Certain materials such as gold, silver, and aluminum, as well as polymers like poly-tetrafluoroethylene (PTFE), exhibit remarkable reflectance properties, particularly in the infrared. Silver is also known for its high visible reflectance qualities as well. Reflectance of 99% in the inner trap surface should, by the ideal conditions mentioned above, bring about a hundred photon paths on average. Small absorption by aerosols can be assumed, for normal circulating air conditions on HVAC systems. All this leads to the conclusion that a significant reduction of escaping light is necessary for the design of a good light trap. Dielectric mirrors and distributed Bragg reflectors, including mirrors fabricated from alternating layers of different refractive indices—can be designed to reflect even 99.9% of a range of wavelengths. These mirrors are also preferable to serve as inner surface coatings for the light traps according to this disclosure.

The current invention aims to disclose several embodiments designed to decrease the light escape from the inlet and outlet apertures. Any combination of these embodiments is meant to be included in the scope of the current invention. Elongation of the main axis of the trap i.e., from the geometric center of the inlet aperture to the one of the outlet aperture, is significant. A high ratio between the main axis length to the apertures' area, should contract the solid angle of the apertures as measured from the middle of the trap. Less light escapes from farther apertures and more reflections on average would be allowed. This elongates the light average path through the trap and increases the chances for the light to be absorbed by an aerosol. A concave curvature can inflect light beams away from the ends of the trap. This feature, which is known for some prior art, works in synergy with the elongation of the main axis. Such concave shapes may be for example of ellipsoidal curvature or formed with a parabolic contour along the main axis. An elongated structure with concave contour and round cross-section i.e., perpendicularly to the main trap axis, is demonstrated in FIG. 1. This oval structure encompasses light sources in about the middle of the main axis. Light is less likely to escape, for the many reflections, on average, it should undergo on its way out. Furthermore, due to the concave shape, any reflection should inflect the light course in a direction closer towards the center of the trap compared to a similar trap with a reflector that is parallel to the main axis.

An additional preferred feature of the current invention is aimed to increase the radiation absorbance of aerosols in the system and refers to the volume of the trap. An increased volume of the trap—larger than the volume of the main axis multiplication by the average area of the apertures, possesses two considerable edges. At first, the high volume slows down the mass flowing velocity through the trap. Therefore, the extra time would make pathogens more prone to deactivation processes. A second advantage lies in the fact that a larger trap allows a higher average distance for a light beam to travel between two reflections. In this way, absorbance in aerosols increases, while absorbance at the inner trap surfaces decreases. In addition to the energy savings from the multiplication of light intensity inside the trap, the current invention does not demand compression of the air by a smaller outlet aperture. For volume increase implementation, a wider cross-section of the trap, than the inlet or outlet apertures can be applied. It may vary along the main axis or remain constant throughout the trap. FIGS. 2 and 3 describe a cylindrical trap with bases, that consists of the ends of the trap which are bigger than the inlet and outlet apertures. This kind of light trap has about a right angle between the aperture plane and the rest of the cylindrical wrap. This architecture allows higher volume, while incident light can reflect from the ends of the trap if it had not to travel directly to the apertures themselves.

Another object of the present invention is to provide a means of blocking radiation from escaping the trap. Such a light shield possesses the ability to reflect incident light, as well as inflecting the incoming or outgoing air around it. A concave reflective surface can allow the light shield to blend as a complementary part of the oval shape of the inner reflective surface of the trap. FIGS. 4 and 5 exhibit instances of such an oval, light-shielded trap. This part combines an aerodynamic shape for the reduction of air resistance through the trap and a concave reflective inner surface for light trapping purposes.

Multiple traps can work together in series according to the current invention. The traps may work as independent units or be inherently combined. Such a combined traps system is demonstrated in FIG. 6. This combination may contain light sources only in the more central section of the system. Hence, placing the source of the light farther from the inlet and outlet apertures. By this method, light still escaping one trap should be circulated again in a neighboring one. This invention is not limited to any combination of inherently connected or separated cleansing trap unites, connected in parallel, in series or incorporates both for HVAC systems. Moreover, as FIG. 7 suggests, different cross-section geometries can be adopted by the trap or by the flange connector or by both. Square or rectangular geometries of the trap, the inlet and outlet apertures or the flange connector itself, may allow the trap to fit easier to the adjacent HVAC duct or the allocated space.

An additional feature of the present invention is the light source(s) spectra. This invention focuses on the infrared and visible spectra. Both light types do not have enough energy to deactivate biological macromolecules by one photon absorption. Yet, the energy of these lights is converted to heat upon absorption by aerosols. Absorption of energy by a pathogen, at a higher rate than energy dissipation to the environment, is the key for disinfection. Therefore, intensified infrared and visible radiation hold added value in regard to sterilization by high field in a light trap, compared to intensified UV light. Light in the infrared and visible range is also not mutagenic. This is important for user safety and to refrain from accelerating mutagenesis by low wavelength radiation among pathogens. Installation of the trap at the endpoint of HVAC system is also possible, due to the safety of low-frequency light. Additionally, the cytotoxic reactive oxygen species synthesis which is triggered by visible light, and water heating and evaporation effects owing to infrared absorption makes these spectra preferable for the current invention. Another embodiment of this invention is the illumination of the light trap by optical fibers. By this method, light can be conveyed from an external light source into the trap. The fiber can be inserted through a special small aperture on the surface of the trap or through the inlet or outlet apertures. Accordingly, light source units and their wiring, will not interrupt the operation of the trap by absorbing a part of the light. Such optical fibers may illuminate sunlight collected by a solar concentrator on its other end. Also, the effective photoinactivation of wide range of viruses was shown to take place in aqueous solutions using high-intensity, low wavelength visible light or sunlight (M. Hessling et al. photonics 2022, 9, 113.). The disclosed light trap can increase the intensity of such light in a water-configured light trap when the fluid is essentially permeable to a substantial part of the irradiated light.

Embodiments as safety of use, low energy consumption, the possibility for installation at the endpoint, and simplicity of design, imply that the technology can be used not only in industrial HVAC systems. Therefore, mobility is an optional feature for the light traps which fits relatively small HVAC systems. It is an additional object of the current invention to disclose a mobile HVAC device for the purification of ambient air. The system comprising a compact light trap, connected to light source(s) as well as a mobile HVAC system. This system may serve as an air purifying ventilation system or alternatively incorporate any heating, air conditioning, or cooling functionalities. Additionally, this system may consume energy from the electric grid or have an independent mobile power source. Small devices can be wearable and may be combined with other wearable gear as protective masks. This can provide personal protection by continuous clean air supply around the user. This wearable system can be extra beneficial during commercial flights for instance. The maintenance of such a device is straightforward as well, with occasional replacement of a light source.

Further operation methods, characteristics, advantages, and functionalities of the current invention will be evident by the following claims and the accompanying drawings. Also, the parts functionality and assembly combinations, as well as elements of manufacture should be more apparent.

### BRIEF DESCRIPTION OF THE FIGURES

The believed key elements of the current invention are provided below by way of example. Emphasis should be placed on the fact that the drawings are presented for illustration and clarification purposes. These are not intended to define the limits of the present invention. Also, the following disclosure of claims will allow a person skilled in the art to manufacture and utilize the present invention. This person will apprehend that variations, modifications, and alternations may be applied, without departing from the scope and spirit of the invention.

FIG. 1 is an illustration of an elongated light trap with a concave contour and rounded cross-section;

FIG. 2 illustrates a cylindrical trap with a diameter wider than its connecting flanges;

FIG. 3 is a sectional view showing the inner parts of the trap in FIG. 2;

FIG. 4 corresponds to a compact light shielded trap;

FIG. 5 describes a compact light shielded trap with flanges to connect an HVAC system;

FIG. 6 shows a trap which is containing multiple concave inner reflective surfaces; and

FIG. 7 illustrates a multiple concave inner reflective surface trap with a square cross-section.

### DETAILED DESCRIPTION OF THE ILLUSTRATED EMBODIMENTS

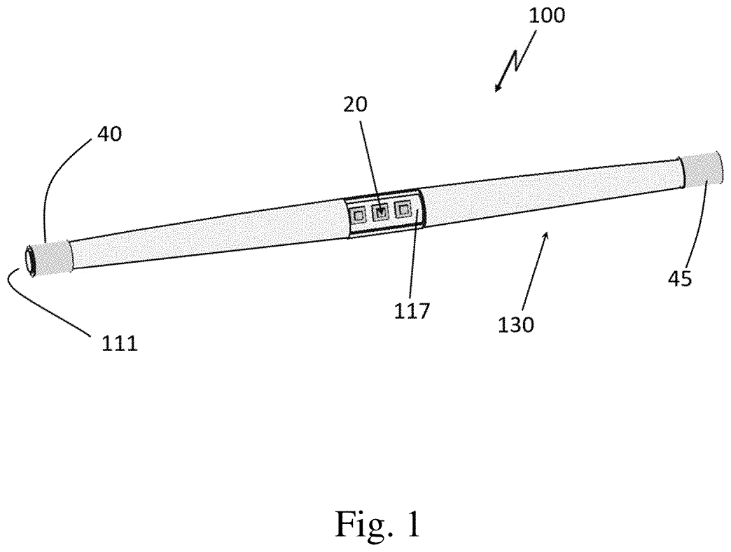

Reference is now made to FIG. 1, which describes an elongated light trap with a concave contour and a round cross-section 100. This trap is compounded from a trap frame 130 which contains an inner reflective surface 117. This surface may be of the same material as the trap frame 130, such as aluminum for instance. Alternatively, a coated reflective surface as silver or dielectric mirror layers may form the inner reflective surface. Additional polymeric or glass transparent coating may cover the reflective inner surface. Moreover, light sources 20 are interconnected to said trap 130. These light sources can be placed at any place inside or on the inner surface of the trap. Yet, a preferred location for the light source(s) should be in about the middle of the trap. The combination of the reflective surface with the elongated geometry of the trap should allow light to be reflected multiple times on average. Inlet flange 40 with an inner diameter of about the inlet aperture 111 and outlet flange 45 are interconnected to trap 100. These flanges should interconnect the trap to the ducts of the HVAC system. Therefore, the trap should be integrated as a part of the HVAC system while disinfecting its flowing air. The cut around the middle of the trap is visible in this sectional view for the illustration of the inner surface and light sources.

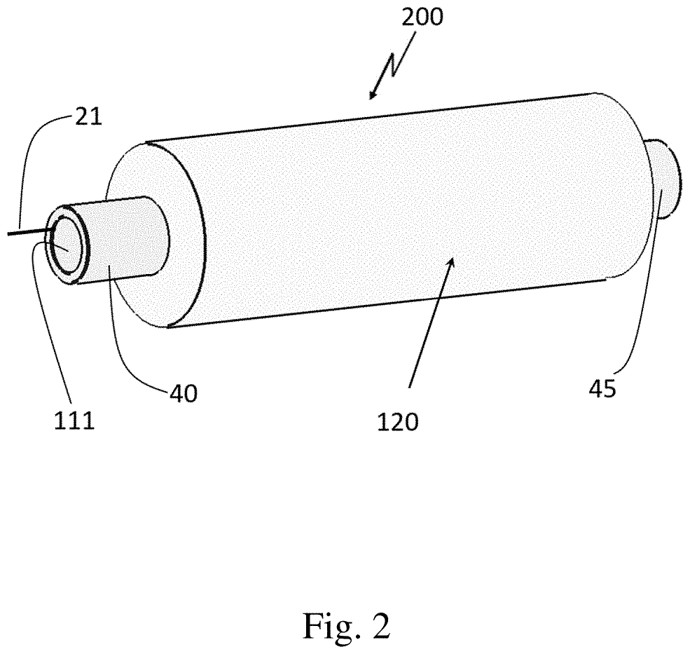

FIG. 2 displays the full cylindrically shaped disinfection light trap 200 with trap frame 120. This trap demonstrates a larger trap diameter than inlet flange 40 and outlet flange 45, although in many cases symmetry may render the two installation directions interchangeable. Said flanges are supposed to be about the diameter of the HVAC ducts, leading the air in and out of the system. By this feature, the volume of the trap should be higher, and the inner surface, on the ends of the trap, next to the apertures can reflect light back as well. Electrical connecting line 21 for the light source(s) is demonstrated here. The inner surface of the trap should contain a duct for the air to flow from the inlet aperture to the outlet aperture, engulfed with reflective material or layered mirror as dielectric or distributed Bragg reflector for most of the inner surface. Optical fibers or other light sources as LED lights, should illuminate the inner trap surface, providing excessive denaturing energy to any pathogens which pass through.

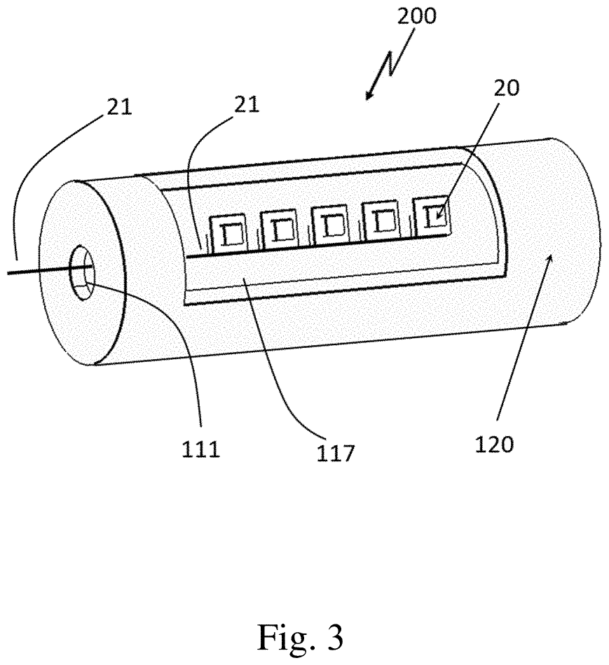

A sectional view of said cylindrically shaped disinfection light trap 200 is depicted in FIG. 3. This visualizes the inner parts of the trap as the reflective inner surface 117 and light sources 20. LED light sources are preferred light sources for this invention for their high-efficiency properties. Another preferred origin of light is directed by optical fibers into the trap. These kinds of lights are widely available and allow efficient infrared and visible light output. Five light sources are connected to light trap 200 in this figure example. These lights can be arraigned differently with a single light or multiple light sources. As an alternative, light source(s) connected to the trap, by means of optical fiber(s) can carry light to the inner space of the trap. This brings unwanted absorption by any lightings installed and their wiring to the minimum.

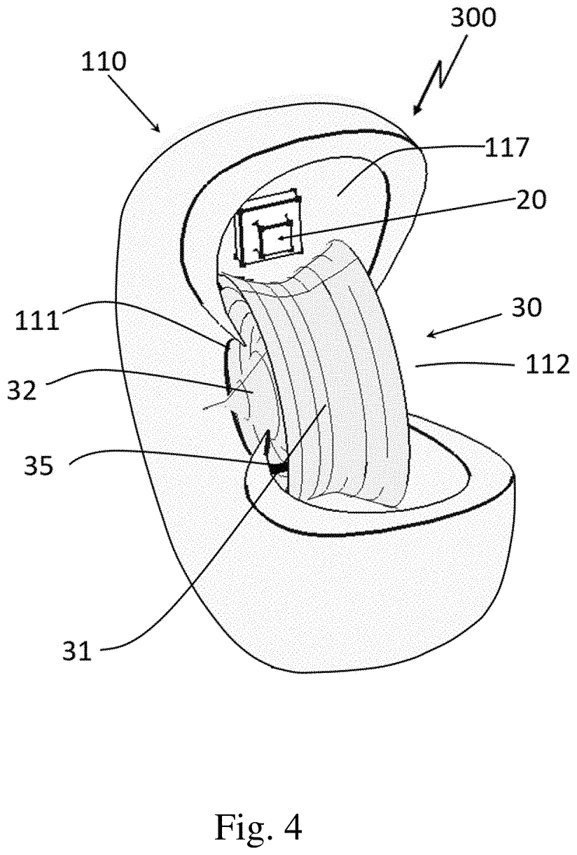

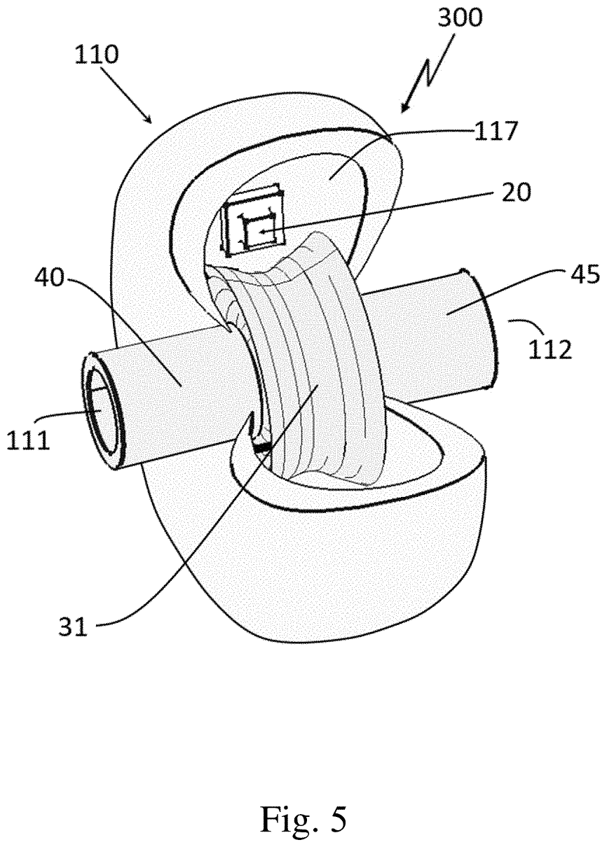

FIG. 4 exhibits a compact light-shielded trap 300 with a trap frame 110. The frame contains a reflective inner surface 117, capable of substantially reflecting the light of light source 20. This trap has an inlet aperture 111 and an outlet aperture 112. The trap is interconnected to light shield 30 by connectors 35. The light shield holds the ability to reflect light at least partially. This is especially from the surfaces facing the inner parts of the trap as surface 31. An optional deflecting section 32 of the shield is aerodynamically designed to allow more linear flow through the light trap. Additional inlet flange 40 and outlet flange 45 are added and shown in FIG. 5. These flanges should allow easier and tighter connections to the HVAC system. Said light-shielded trap 300 may be elongated as well and might also be shaped in other oval or cylindrical shapes. In this case, the light shield 30 may be adjusted to the overall length of the trap. Alternatively, two separated light shields can cover the inner aperture and outlet aperture.

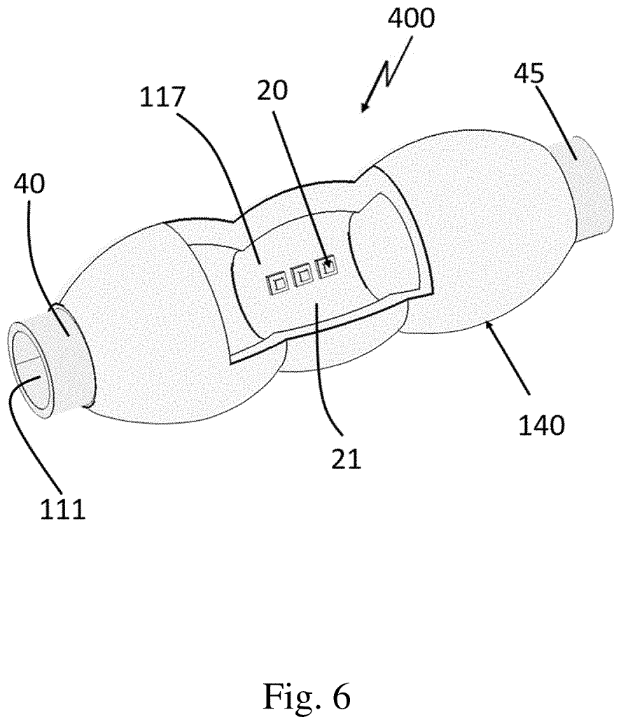

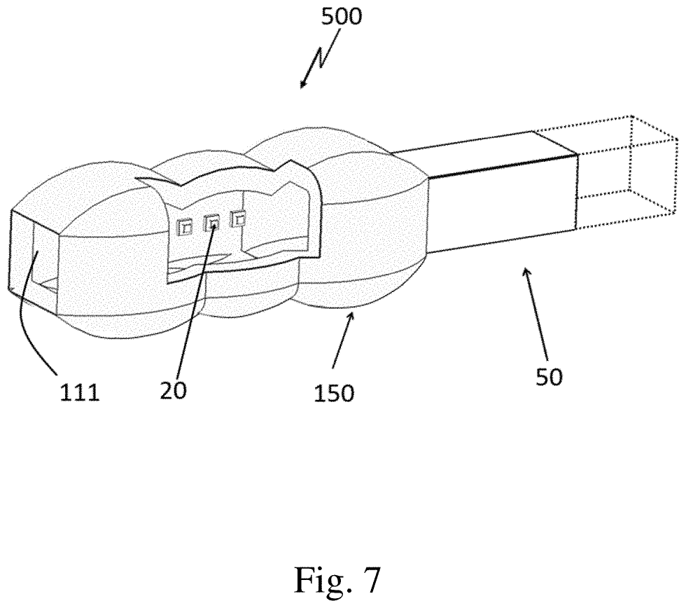

Multiple concave inner trap surfaces are demonstrated in FIG. 6. This light trap 400 with trap frame 140 contains three light-reflective compartments. Each of these acts as a separated active unit, reflecting light from its inner surface 117. Escaping light now may circulate through the adjacent traps. By this method, not all the compartments must contain a light source. This trap sample contains three light sources 20 and electrical wiring 21. Such a trap and all other trap types can be shaped in different cross-sectional geometries as well. FIG. 7 illustrates a square resembling cross-sectional design. This light trap 500 with trap frame 150 can fit better to a square duct of HVAC system partially drawn as part 50. Clearly, it is possible that only the inlet and outlet apertures or the connecting flanges will be shaped according to the HVAC duct. In trap 500, the trap's body is squarely designed too. This may fit better to the allocated volume of the HVAC system and turn the airflow from a square duct, more linear in nature.

What is claimed is:

1. An air purification system comprising at least:
a. a light trap comprising: a frame that forms an air duct connecting an inlet and outlet apertures, which allows air to flow through, and an inner reflective surface;
wherein the light trap possesses at least one of the following properties:
i. a concave curvature geometry for most of the inner surface area;
ii. a larger inner volume than the multiplication of the length between the two apertures by the average area of the inlet and outlet apertures;
wherein said light trap's inner reflective surface possesses the attribute of at least 80% reflectance of incident light intensity for at least a part of the infrared or visible ranges or at parts of both infrared and visible ranges;
b. at least one light source, wherein most of its light intensity is in the form of visible, or infrared, or both visible and infrared light;
wherein:
said light source can illuminate the inner reflective surface of the light trap;
and the visible and infrared part of the light emitted by said light source (b) can be substantially reflected by the inner reflective surface of said light trap (a).

2. The air purification system of claim 1, wherein the inner reflective surface of the light trap is made of a material or a compound containing any material from the following group: aluminum, silver, copper, gold, PTFE, polyethylene (PE), polypropylene, polyethylene terephthalate (PET), biaxially-oriented polyethylene terephthalate (BoPET), and polyvinyl chloride (PVC); wherein, an optional substantially transparent coating may cover said reflective surface.

3. The air purification system of claim 1, wherein the inner reflective surface of the light trap is a dielectric mirror or a distributed Bragg reflector, including mirrors fabricated from alternating layers of different refractive indices.

4. The air purification system of claim 1, wherein the light trap further comprises an inlet flange and an outlet flange, capable of connecting the light trap as a part of the airflow duct of an HVAC system.

5. The air purification system of claim 1, wherein said light source is in the form of optical fibers light source or include optical fibers, which illuminate the trap with light that includes infrared radiation or visible radiation, or both types, by an external light source, optically connected to said optical fibers.

6. The air purification system of claim 1, wherein said system further comprising at least one light shield in front of any of the light trap's apertures.

7. The air purification system of claim 1, wherein said air purification system is further configured as a part of an HVAC system, in any part throughout the HVAC system or connected in series to the HVAC system, its ends or an extension duct of the HVAC system.

8. The air purification system of claim 1, wherein said air purification system is further configured to fit as a part of an HVAC system of an airplane, a cruise ship, a bus, a car, or a train.

9. The air purification system of claim 1, wherein said light trap is illuminated by essentially visible light sources only.

10. The air purification system of claim 1, wherein said system contains more than one of said light trap, which are interconnected in series, in parallel or in a combination of both fashions.

11. The air purification system of claim 1, wherein at least one of said light sources is in the form of a solar concentrator capable of conveying sunlight into said light trap via a duct with an inner reflective surface or through optical fibers.

12. A portable air purification HVAC unit comprising at least:
a. an air purification system according to claim 1;
b. a portable HVAC system, capable of any full or partial combination of the following features: ventilating, heating, cooling, and air conditioning;
wherein: said air purification system is connected to said portable HVAC system, so that the air which goes through the portable HVAC system travel at least partially through the air purification system too.

13. The portable air purification HVAC unit of claim 12, wherein said portable unit is further configured as a wearable device, capable of supplying purified air around the respiratory tract of the user.

14. A fluid purification system comprising at least:
a. a light trap comprising: a frame that forms a duct connecting an inlet and outlet apertures, which allows fluid to go through, and an inner reflective surface;
wherein the light trap possesses at least one of the following properties:
i. a concave curvature geometry for most of the inner surface area;
ii. a larger inner volume than the multiplication of the length between the two apertures by the average area of the inlet and outlet apertures;
wherein at least a part of said light trap's inner reflective surface possesses the attribute of substantial reflectance of incident light for at least a part of the infrared or visible ranges or at parts of both infrared and visible ranges;
b. at least one light source, wherein most of its light intensity is in the form of visible, or infrared, or both visible and infrared light;
wherein:
said light source can illuminate the inner reflective surface of the light trap;
and the visible and infrared part of the light emitted by said light source (b) can be substantially reflected by the inner reflective surface of said light trap (a).

15. The fluid purification system of claim 14, wherein said light source emits exclusively light with wavelengths exceeding 450 nm.

16. The fluid purification system of claim 14, wherein said fluid purification system is further configured as a part of a water purification system, an aqueous solutions purification system, or as an HVAC system, in any part throughout the HVAC system or connected in series to the HVAC system, its ends or an extension duct of the HVAC system.

17. The fluid purification system of claim 14, wherein said light source emit light primarily in the visible spectrum and no other light source illuminates the inner surface of said light trap with another kind of electromagnetic radiation.

18. The fluid purification system of claim 14, wherein said system is further configured to purify passing air, water, or aqueous solutions.

19. The fluid purification system of claims 14, wherein at least one of said light sources is in the form of a solar concentrator capable of conveying sunlight into said light trap via a duct with an inner reflective surface or through optical fibers.

20. The fluid purification system of claim 19, wherein said light trap further comprising a substance capable of a photocatalytic activity with the absorption of sunlight.
